(19) United States
(12) Patent Application Publication
(10) Pub. No.: US 2018/0114593 A1
(43) Pub. Date: Apr. 26, 2018

(54) PATIENT LIVING RECORD

(71) Applicant: HAWTHORNE EFFECT, INC., Alamo, CA (US)

(72) Inventor: Jodi J. AKIN, Alamo, CA (US)

(21) Appl. No.: 15/565,595

(22) PCT Filed: Apr. 8, 2016
(86) PCT No.: PCT/US16/26694

§ 371 Date: Oct. 10, 2017

Related U.S. Application Data
(60) Provisional application No. 62/146,239, filed on Apr. 10, 2015.

(51) Int. Cl. G16H 10/60; G16H 10/20

(52) U.S. Cl. CPC G16H 10/60; G06Q 50/10; G16H 10/20

(57) ABSTRACT

A system and method is provided for a patient-driven platform that generates a comprehensive, living health biography of the patient, where patients control access or visibility to aspects or portions of the information. A patient's autobiographical information and medical information is received via a graphical user interface (GUI) from a user. Access and permission levels are assigned to the inputted information as specified by the user. The patient's information is updated with continuous variable data being monitored by the patient. When a request for patient information is received from a requestor, a determination is made if the requestor is authorized to access the patient's information. In the event the requestor is authorized, then access is granted and the type and level of information that is provided is based on the user-assigned access and permission levels.

### CROSS REFERENCE TO RELATED APPLICATIONS

This application claims priority benefit of U.S. Provisional Application Ser. No. 62/146,239 filed 10 Apr. 2015; the contents of which are hereby incorporated by reference.

### FIELD OF THE INVENTION

The present invention in general relates to the field of clinical trials and medical record keeping and in particular to an automated platform for a patient driven living health biography.

### BACKGROUND OF THE INVENTION

Clinical trials are prospective biomedical or behavioral research studies on human subjects that are designed to answer specific questions about biomedical or behavioral interventions, and generate safety and efficacy data. Examples of clinical trials include trials for novel vaccines, drugs, treatments, devices, or new ways of using known interventions. Clinical trials are conducted only after satisfactory information has been gathered that satisfies health authority and ethics committee approval in the country where approval of the therapy is sought.

Depending on product type and development stage, investigators initially enroll volunteers and/or patients into small pilot studies, and subsequently conduct progressively larger scale comparative studies. As positive safety and efficacy data are gathered, the number of patients typically increases. Clinical trials can vary in size, and can involve a single research entity in one country or multiple entities in multiple countries.

Clinical trials can be quite costly, with the costs of clinical trials having nearly doubled in the last decade. In fact, a full series of trials may cost hundreds of millions of dollars with the burden of paying usually borne by the sponsor, which may be a governmental organization, an investor or investment fund, a pharmaceutical, biotechnology or medical device company. In addition hospitals may pay to participate in a clinical trial in order to be first in line for treatment options for patients. When the required support exceeds the sponsor's capacity, the trial may be managed by an outsourced partner, such as a contract research organization or an academic clinical trials unit.

Besides the great costs associated with clinical trials, clinical trials may be quite lengthy and may last several years. There are many reasons why clinical trials can be so lengthy including that for chronic conditions such as cancer, it takes months, if not years, to see if a cancer treatment has an effect on a patient. For drugs that are not expected to have a strong effect (meaning a large number of patients must be recruited to observe ‘any’ effect), recruiting enough patients to test the drug's effectiveness (i.e., getting statistical power) can take several years. Furthermore, only certain people who have the target disease condition are eligible to take part in each clinical trial. Researchers who treat these particular patients must participate in the trial. Then researchers must identify the desirable patients and obtain consent from the patients or their families to take part in the trial. Finally, the biggest barrier to completing studies is the shortage of people who take part. All drug and many device trials target a subset of the population, meaning not everyone can participate. Some drug trials require patients to have unusual combinations of disease characteristics. It is a challenge to find the appropriate patients and obtain their consent, especially when the patients may receive no direct benefit (because the patients are not paid, the study drug is not yet proven to work, or the patient may receive a placebo). In the case of cancer patients, fewer than 5% of adults with cancer will participate in drug trials.

Additional problems associated with carrying out clinical trials on a timely and effective basis include lost documentation or incomplete or poor patient follow-up, incomplete tests, poor quality of collected data, and lack of sufficient evidence to drive decisions. This leads to frustration and impedes timely approvals of novel therapies, which slows innovation and adds costs to sponsors, regulators, and ultimately consumers and the healthcare system overall. In addition, delayed and ineffective trials lower investment returns to sponsors and investors.

Thus while clinical trials can be an effective tool, there exists a need for a method and system to improve the implementation of clinical trials by improving patient interaction and follow-up, data collection, and information processing in order to lower costs and cycle times of clinical trials.

### SUMMARY OF THE INVENTION

A non-transitory computer-readable medium for implementing an automated patient-driven living health biography is provided that has instructions stored thereon, that when executed on a processor provides a graphical user interface (GUI) from a platform. A patient's autobiographical information and medical information is received via the graphical user interface (GUI) from a user. Access and permission levels are assigned to the inputted information as specified by the user. The patient's information is updated with continuous variable data being monitored by the patient. When a request for patient information is received from a requestor, a determination is made if the requestor is authorized to access the patient's information. In the event the requestor is authorized, then access is granted and the type and level of information that is provided is based on the user-assigned access and permission levels. A system and process for operating such a system is also provided.

### BRIEF DESCRIPTION OF THE DRAWINGS

The present invention is further detailed with respect to the following drawings. These figures are not intended to limit the scope of the present invention but rather illustrate certain attributes thereof.

FIG. 1 is a screenshot of a home page for an embodiment of the App or program for the personal health diary and data collection system according to an embodiment of the invention;

FIG. 2 illustrates an exemplary workflow for the registration and establishment of an ongoing patient medical history according to an embodiment of the invention;

FIG. 3 is a flowchart of a method and workflow of a trial using the clinical trial platform and personal health diary and data collection system according to an embodiment of the invention; and

FIG. 4 is a schematic diagram illustrating an overall view of communication devices, computing devices, and mediums for implementing embodiments of the invention.

### DESCRIPTION OF THE INVENTION

The present invention has utility as an automated system and method to improve the implementation of clinical trials and general patient medical care by improving patient data collection and record keeping. Embodiments of the inventive method leverage the concept called “The Hawthorne Effect”. The Hawthorne Effect is a psychological phenomenon that produces an improvement in human behavior or performance as a result of increased attention or observation.

While several steps performed by an inventive process and a non-transitory system involve one or more abstract ideas, the present invention as a whole includes aspects that amount to significantly more than the abstract idea. Indeed, aspects of the present invention have never been previously performed in manual or digital form prior to the present invention. As recitations of using a non-transitory computer medium to compile a stored list of user inputted and automatically generated information that may be derived from wearable technology and monitoring devices to form a comprehensive, patient-driven living health biography, where patients control access or visibility to aspects or portions of the information submitted. These aspects of the present invention as a data collection or repository amount to significantly more than simply organizing and comparing data by addressing the challenges of tracking of patient condition and response to medical or surgical treatments, which is essential to the entire healthcare ecosystem. As a result, the present invention provides a patient-driven platform that generates a comprehensive, living health biography of the patient, where patients control access or visibility to aspects or portions of the information. Through embodiments of the platform, a conversational, ongoing, curated and dynamic recording of a patient history is obtained. The present invention has the potential effect of improving human behavior or performance as a result of increased attention or observation.

With respect to the 2014 Interim Eligibility Guidance, the claimed invention is not directed to an abstract idea. The method claims recite a series of acts for implementing an automated patient-driven living health biography and monitoring application (app) on an end user device to form a comprehensive, patient-driven living health biography, where patients control access or visibility to aspects or portions of the information submitted to a server, that are executed on a processor. Thus, the claim is directed to a process, which is one of the statutory categories of invention (Step 1: YES).

Analysis of the claim may then be conducted to determine whether the claim is directed to any judicial exception. The claimed invention relates to software technology that implements an app on an end user device with a graphical user interface (GUI) from a platform, where the platform receives a patient's autobiographical information and medical information, which may be derived from wearable technology and monitoring devices, via the graphical user interface (GUI) from a user, assigns access and permission levels to the inputted information as specified by the user, and updates the patient's information with continuous variable data being monitored by the patient. The platform receives requests for patient information from requestors, determines if the requestor is authorized to access the patient's information, and if the requestor is authorized, then access is granted and the type and level of information that is provided is based on the user-assigned access and permission levels. Such action does not describe an abstract concept, or a concept similar to those found by the courts to be abstract, such as a fundamental economic practice, a method of organizing human activity, an idea itself (standing alone), or a mathematical relationship. In contrast, the invention claimed here is directed towards and relates to software technology that performs an automated patient-driven living health biography and monitoring application (app) on an end user device with a graphical user interface (GUI) from a platform to form a comprehensive, patient-driven living health biography, where patients control access or visibility to aspects or portions of the information submitted to a server, a concept inextricably tied to computer technology and distinct from the types of concepts found by the courts to be abstract. Accordingly, the claimed steps do not recite an abstract idea. Nor do they implicate any other judicial exception. Accordingly, the claim is not directed to any judicial exception (Step 2A: NO). The claim is eligible.

In addition, the claimed invention is also directed to a non-transitory computer-readable medium, which is a manufacture, and thus a statutory category of invention.

In embodiments of the invention, data in a patient biography is obtained and updated from two sources or domains: patient-driven autobiography supplied by the patient or obtained from electronic medical records and continuous variables that may be derived from wearable technology and monitoring devices.

Patient-driven autobiography may be broken up or organized into major categories that may illustratively include: life stats; religious or cultural health beliefs; diet and exercise profiles; medications; labs; major social/life milestone events; medical visit findings; imaging; and diary. Life stats may illustratively include one or more of biometric identifiers such as fingerprints, retinal scans, etc.; birth statistics such as date of birth, birth weight, location of birth, etc.; family history which may illustratively include information on parents and siblings, diseases and causes of death, genetic markers, etc.; immunization records; medical milestones such as surgeries, pregnancies, hospitalizations, diagnoses, dental records, allergies, etc.; religious or cultural health beliefs may illustratively include a list of observances and restrictions especially with relation to medical care and diet; diet and exercise may illustratively include dietary restrictions and food allergies, food preferences, vegetarian, vegan, exercise routine, etc.; medications may illustratively include the reasons for taking the medications, the dates taken, and dosages; labs may illustratively include laboratory diagnostic test results such as for blood counts and other test levels; major social/life milestone events may illustratively include relationship (married/single), education (level achieved), vacations, family milestones, etc.; medical visit findings summarizes when and what was accomplished at various doctor office or clinic visits; imaging is a collection of available scans of the body including x-ray, computed tomography (CT), magnetic resonance imaging (MRI), breast exams, etc.; and diary is a repository of general thoughts and feelings of a patient, and may be useful for psychological profiling and analysis.

Continuous variables may illustratively include weight, height, vital signs, electrocardiogram (EKG or ECG), and wellbeing (quality of life survey(s)). The continuous variables may be obtained, in real time or recorded for later download, from wearable devices such as watches, smartphones, portable computing devices, etc. The wearable devices may be wired or wirelessly connected to the patient's mobile phone, tablet, laptop, or desktop computer for storing the recorded information. In a specific embodiment, the information may be stored in a patient account in the cloud on a remote server.

A graphical user interface (GUI) is provided in embodiments of the invention for the patient to input their information and may include drop-down menus and finable forms and questionnaires. In addition, data may be collected, coded, and stored through voice, video, and interview mechanisms, as well as options for “form” style. Dashboards and dynamic graphics may be designed for displaying dynamic information of interest, illustratively including blood pressure history, weight/body mass index (BMI) history, key lab history, and other parameters of interest. Custom records/reports may be generated and provided to the patient who may make the records/reports available to selected stakeholders (doctors, medical personnel, family, caregivers, etc.).

In embodiments of the inventive patient-driven living health biography, a patient (or their designee) may select which sections or fields are shared, and which fields are protected. Data is kept on a secure server environment where health care providers have access based upon permissions selected by the patient. If a patient participates in a clinical trial, the record will become available to the study database environment based upon protocol requirements.

Embodiments of the patient-driven living health biography may be configured as a program for a computer, or an App (application) for a mobile device such as a smartphone, tablet, or wearable. Proprietary versions of the patient-driven living health biography software may be configured for clinical trials.

In specific embodiments of the App and program, a personal health diary and data collection system is provided for recording prior health events, present health events and related follow-up visits. The patient living history may be cloud-based with records stored in a multi-tenant database, where the records are secure, private, and confidential. The records may form a patient living history from birth to the present moment in the life of the patient. The living history is recorded in a novel way, with the patient made to feel as if they are a person rather than a “subject”. The patient living record may be used as a standalone tool for patients and as a tool for patient assessments that may be utilized to provide important follow-up data to primary care teams, hospitals, and research organizations.

Embodiments of the App and program are configured to allow interaction, documentation and verification of certain medical data points by a health care provider, while also providing the patient the ability to have their complete health history in one cloud-based location, Embodiments of the App and program are intuitive, easy to update, and provide an audit trail complying with accepted clinical research practices. Embodiments of the App and program provide reporting/graphics that allow a patient to see progress on health initiatives, show trends in outcomes, generate prompts for follow-up visits. and access health information. Patients using the App and/or grogram have the. ability to share all or part of their health records privately with any health care professional of their choosing, with their entire healthcare team, or with specific concerned parties—from a primary doctor to a chiropractor, yogi, children, etc. The App and program saves a patient the effort of repeatedly completing new patient forms or worrying about details they may have forgotten over time. Embodiments of the App and program may be configured for audio and video interviews with patients and/or with health care providers. The App and program may also provide a single universal format for documenting patient visits, where only a patient and the health care provider may enter/audit data, and offers a convenient platform when participating in clinical research or health surveys, while allowing for patient surveys and assessment forms to be added as needed for patient-tailored assessments or participation in clinical trials.

Embodiments of the App and program may be built Oil programming standards for integration with existing health system electronic health records, and is compatible with existing software operating systems (OS) for mobile devices illustratively including Microsoft Windows, Apple iOS, and Android platforms, as well as operating systems for desktop and laptop computers illustratively including Microsoft Windows, Apple iOS, and Linux. Embodiments of the App and program may be implemented. with an electronic data capture (EDC) system, which is a computerized system designed for the collection of clinical data in electronic format for use in human clinical trials.

Security in specific embodiments of the App and program may augment traditional password access with additional layers of security, illustratively including biometric access (fingerprint, retinal scan, voice pattern recognition, handwriting analysis). Security levels may also be configured to allow second and third party access privileges. In all instances, access to patient data and information is compliant with the Health Insurance Portability and Accountability Act (HIPAA). Data storage in specific embodiments is keyword and image searchable, and archives image, text, and voice records by date, time, and event.

The interface used in specific embodiments of the App and program provides a graphical user interface (GUI) that is based on intuitive screens/tabs with a uniform look and feel (screen colors, logos, layout). Common control gestures such as slide and touch for touch screen displays, as well as voice commands and traditional mouse pointers and mouse pads may be used to make selections. Additional features that may be provided in specific embodiments of the App or program may illustratively include voice interactive data capture, live Chat, video conferencing, conversation recordation, audit trail for designated sections, and social media access.

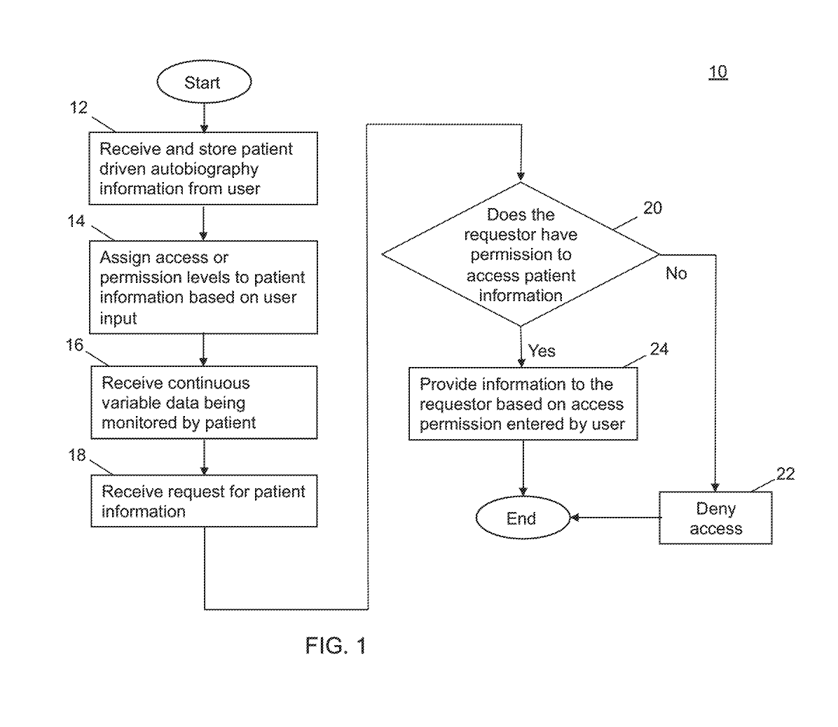

Referring now to the figures, FIG. 1 illustrates an example of a home page 10 for an embodiment of the App or program for the personal health diary and data collection system with a series of tabs or buttons for entering or recalling previously stored information. It is appreciated that—depending on the size of the display of the user device—more or fewer tabs may appear on the screen. In addition, some tabs may be customizable based on the required application or customer for an embodiment of the inventive software. Illustrative examples of screens that may be accessed based on selections of tabs or buttons include: My beginnings (when and where born, details of birth, health issues at birth), My family history (health survey of family members, genetic information, cancer risk profile), My medications (drugs taken, including date taken, dosages, side effects), M chronic illnesses, My vaccination/immunization history, My hospitalizations (visits over 24 hours—when, why, discharge status, emergency room (ER) visits when, why, discharge status), My images (X-ray, CT scans, Angios, Ultrasounds, MRIs—when and why), My doctor office visits, Dental history (visit history/habits, dentures/implants, periodontal disease), My healthcare family (team) (primary physician, specialists, dentist, favorite nurses, special services—physical therapy, chiropractor, acupuncturist, mental health professional), My travel diary, Health care surveys, Religious/cultural healthcare considerations, Lifestyle survey, Search engines, Diary, and Photo gallery.

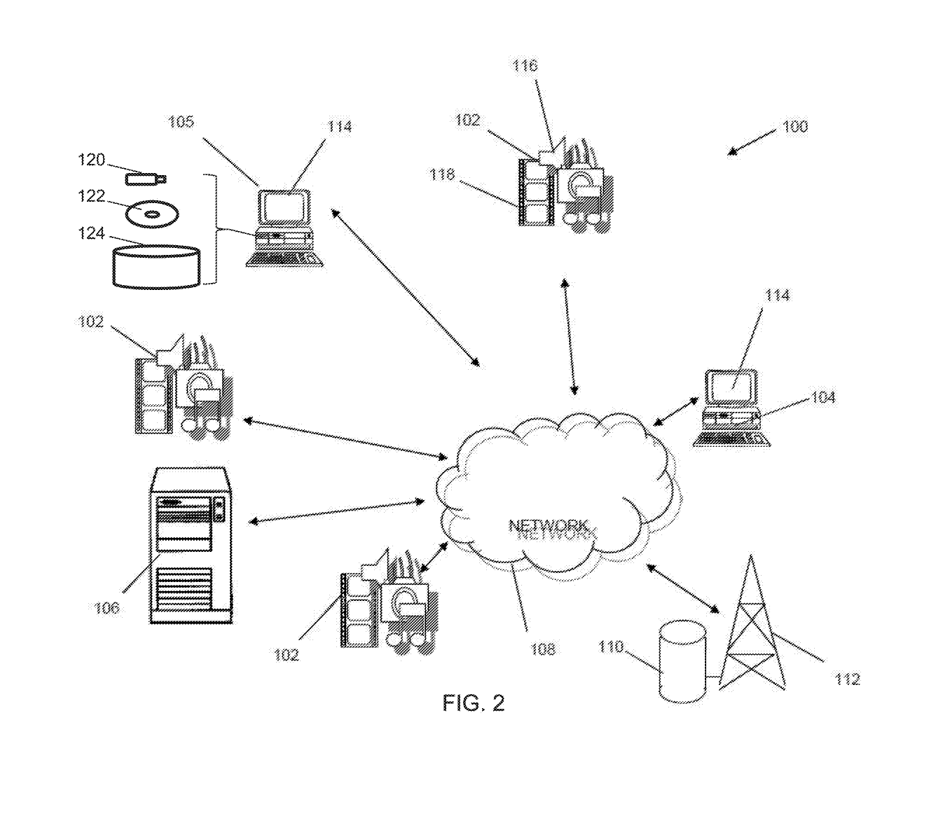

FIG. 2 illustrates an exemplary workflow 20 for the registration and establishment of an ongoing patient medical history. At an initial registration of a patient, the patient is assigned a unique patient identifier and an access code that are generated by the system software (Block 22). Following the registration of the patient, a patient interview is conducted by a health care professional (HCP) where patient vitals may be measured and information about the patient is collected (Block 24). Based on the interview and supplied information, fixed patient record fields are verified by source documentation, and verified information is locked and is no longer updatable (Block 26). Subsequently, patients may update their vital signs (blood pressure, blood sugar levels, temperature) using curated random auto check-up with a snapshot of their physical wellbeing or state (Block 28). Continuous patient monitoring of vital signs and parameters—such as weight, height, vital signs, electrocardiogram (EKG or ECG), and wellbeing (quality of life survey(s))—provides a series of time-stamped data points, where the data points may also be used in an audit trail (Block 30). A patient may also be visited at their residence or come in for a check-up to update the patient status using unique forms (Block 32).

A process and workflow chart 40 for using the embodiments of the inventive clinical trial platform and personal health diary and data collection system is illustrated in the FIG. 3 as follows. A user such as a patient (or their designee) inputs the patient's autobiographical information and medical information via a graphical user interface (GUI) with the patient-driven living health biography platform (Block 42). The user assigns access and permission levels to the inputted information (Block 44). The platform continues to update the user's information including continuous variable data being monitored by the patient (Block 46). The platform receives a request for patient information from a requestor such as a medical professional or caregiver (Block 48). The platform determines if the requestor is authorized to access the patient's information (Decision block 50). If the requestor is not authorized (Decision block 50 is No) access is denied (Block 52). If the requestor is authorized (Decision block 50 is Yes) access is granted and the type and level of information that is provided is based on the user-assigned access and permission levels (Block 54).

An additional suite of Apps and programs are available for health care professionals that may interact with and supplement the personal health diary and data collection system Apps and programs described above for use by and with patients. A health care professional scheduling and professional profile manager program and App provides these illustrative examples of screens and features that may be accessed based on selections of tabs or buttons including : a professional profile, a personal profile, a professional photo, a credentials file, a chatting feature, a scheduling feature, a diary feature, a charting of activities, a link to maps for directions, a link to transportation options, a message board, and professional education links.

A list of information items that may be supplied by a health care provider in their personal and professional profile may illustratively include:
Name (first, middle, last)
Date of birth
Address (street, city, state, country, zip)
Gender
Phone (cell)
Email addresses
Twitter
Instagram
LinkedIn profile
Languages spoken (check minimal, moderate, fluent, written, spoken)
Education history
Degrees (select from drop-down list)
Certifications (select from drop-down list)
Years in clinical practice (provide range)
Therapeutic area experience (select from drop-down list)
Education
Resume/CV builder
Subject matter expertise
Patient care environments worked in (clinic, hospital, ER, OR, ICU, CCU, etc.)
Countries worked in
Skill checklist (blood draw, ECG, echo, etc)
Why you became HCP
Tell a patient story (patient and provider perspective)
Tell a patient story (HCP as patient perspective)
Tell a patient story (HCP as family member/loved one perspective)
Tell a patient story
What you love most about health care professional
What you like least about being a healthcare professional
Something I would like people to know about me
My bedside manner is . . .
Salary range (current)
Ideal hourly rate
Non healthcare skills, talents, interests
My professional bucket list—List of desired accomplishments
My personal bucket list—List of desired accomplishments

A scheduling App and program may be used by the health care provider to schedule and attend to patient requests and needs based on the personal health diary and data collection system Apps and programs described above. The scheduling App and program has a list of locations based on geographical coordinates for health care providers that is also displayed in map form that provides health care professionals that are in close proximity to a patient to be serviced.

FIG. 4 is a schematic diagram illustrating an overall view of communication devices, computing devices, and mediums for implementing the platform for patient-driven living health biography, where patients control access or visibility to aspects or portions of the information according to embodiments of the invention.

The system 100 includes multimedia devices 102 and desktop computer devices 104 configured with display capabilities 114 and processors for executing instructions and commands. The multimedia devices 102 are optionally mobile communication and entertainment devices, such as cellular phones, tablets, and mobile computing devices that in certain embodiments are wirelessly connected to a network 108. The multimedia devices 102 typically have video displays 118 and audio outputs 116. The multimedia devices 102 and desktop computer devices 104 are optionally configured with internal storage, software, and a graphical user interface (GUI) for carrying out elements of the platform according to embodiments of the invention. The network 108 is optionally any type of known network including a fixed-wire line network, cable and fiber optics, over-the-air broadcasts, satellite 120, local area network (LAN), wide area network (WAN), global network (e.g., Internet), intranet, etc. with data/Internet capabilities as represented by server 106. Communication aspects of the network are represented by cellular base station 110 and antenna 112. In a preferred embodiment, the network 108 is a LAN and each remote device 102 and desktop device 104 executes a user interface application (e.g., Web browser) to contact the server system 106 through the network 108. Alternatively, the remote devices 102 and 104 may be implemented using a device programmed primarily for accessing network 108 such as a remote client.

The software for the platform, in embodiments of the invention, may be resident on a widget (USB thumb or flash drive) 120, CD or DVD 122, or an external hard drive 124 for connection to desktop or laptop computers 104, or stored within the server 106 or cellular base station 110 for download to an end user. Server 106 may implement a cloud-based service for implementing embodiments of the platform with a multi-tenant database for storage of separate client data on the platform.

The foregoing description is illustrative of particular embodiments of the invention, but is not meant to be a limitation upon the practice thereof. The following claims, including all equivalents thereof, are intended to define the scope of the invention.

1. A non-transitory computer-readable medium for implementing an automated patient-driven living health biography, comprising instructions stored thereon, that when executed on a processor, perform the steps of:
providing a graphical user interface (GUI) from a platform;
receiving a patient's autobiographical information and medical information via said graphical user interface (GUI) from a user;
assigning access and permission levels to the inputted information as specified by the user; and
updating the patient's information with continuous variable data being monitored by the patient;
receiving a request for patient information from a requestor;
determining if the requestor is authorized to access the patient's information; and
wherein if the requestor is authorized then access is granted and the type and level of information that is provided is based on the user-assigned access and permission levels.

2. The non-transitory computer-readable medium of claim 1 wherein said continuous variables comprise one or more of: weight, height, vital signs, electrocardiogram (EKG or ECG), and wellbeing (quality of life survey(s)).

3. The non-transitory computer-readable medium of claim 1 wherein said continuous variables are obtained, in real time or recorded for later download, from one or more wearable devices, said one or more wearable devices comprising watches, smartphones, and portable computing devices.

4. The non-transitory computer-readable medium of claim 1 wherein said GUI is configured for use on a mobile phone, tablet, laptop or desktop computer of the patient.

5. The non-transitory computer-readable medium of claim 1 wherein said GUI further comprises one or more drop-down menus and a set of fillable forms and questionnaires.

6. The non-transitory computer-readable medium of claim 1 wherein said GUI further comprises dashboards and dynamic graphics designed for displaying dynamic information of interest, said displayed dynamic information comprising one or more of blood pressure history, weight/body mass index (BMI) history, key lab history, and other parameters of interest.

7. The non-transitory computer-readable medium of claim 1 further comprising generating custom records and reports.

8. The non-transitory computer-readable medium of claim 1 wherein the autobiographical information is organized into a set of categories, said set of categories comprising life stats; religious, cultural health beliefs; diet and exercise profile; medications; lab tests; major social and life milestone events; medical visit findings; medical imaging; and diary.

9. The non-transitory computer-readable medium of claim 8 wherein said life stats comprise one or more of: biometric identifiers, family history, and birth statistics; and wherein said biometric identifiers comprise at least one of fingerprints and retinal scans, and said birth statistics comprise at least one of date of birth, birth weight, and location of birth.

10. The non-transitory computer-readable medium of claim 9 wherein said family history comprises one or more of: information on parents and siblings, family diseases, causes of death, and genetic markers.

11. An automated method for implementing a patient-driven living health biography, said method comprising:
providing a graphical user interface (GUI) from a platform;
receiving a patient's autobiographical information and medical information via said graphical user interface (GUI) from a user;
assigning access and permission levels to the inputted information as specified by the user; and
updating the patient's information with continuous variable data being monitored by the patient. receiving a request for patient information from a requestor;
determining if the requestor is authorized to access the patient's information; and
wherein if the requestor is authorized then access is granted and the type and level of information that is provided is based on the user-assigned access and permission levels.

12. The method of claim 11 wherein said continuous variables comprise one or more of: weight, height, vital signs, electrocardiogram (EKG or ECG), and wellbeing (quality of life survey(s)).

13. The method of claim 11 wherein said continuous variables are obtained, in real time or recorded for later download, from one or more wearable devices, said one or more wearable devices comprising watches, smartphones, and portable computing devices.

14. (canceled)

15. (canceled)

16. The method of claim 11 wherein said GUI further comprises dashboards and dynamic graphics designed for displaying dynamic information of interest, said displayed dynamic information comprising one or more of blood pressure history, weight/body mass index (BMI) history, key lab history, and other parameters of interest.

17. (canceled)

18. The method of claim 11 wherein the autobiographical information is organized into a set of categories, said set of categories comprising life stats; religious, cultural health beliefs; diet and exercise profile; medications; lab tests; major social and life milestone events; medical visit findings; medical imaging; and diary.

19. (canceled)

20. (canceled)

21. A system for implementing a patient-driven living health biography, said system comprising:
a server connected via a network to user devices for use by a series of users;
a memory system in electrical communication with said server containing a machine readable medium having stored thereon one or more sequences of instructions which, when executed by a processor, cause a method to be carried out, the method comprising:
providing a graphical user interface (GUI) from a platform;
receiving a patient's autobiographical information and medical information via said graphical user interface (GUI) from a user;
assigning access and permission levels to the inputted information as specified by the user; and
updating the patient's information with continuous variable data being monitored by the patient.

22. The system of claim 21 wherein said continuous variables comprises one or more of: weight, height, vital signs, electrocardiogram (EKG or ECG), and wellbeing (quality of life survey(s)).

23. The system of claim 21 wherein said continuous variables are obtained, in real time or recorded for later download, from one or more wearable devices, said one or more wearable devices comprising watches, smartphones, and portable computing devices.

24. (canceled)

25. (canceled)

26. The system of claim 21 wherein said GUI further comprises dashboards and dynamic graphics designed for displaying dynamic information of interest, said displayed dynamic information comprising one or more of blood pressure history, weight/body mass index (BMI) history, key lab history, and other parameters of interest.

27. (canceled)

28. The system of claim 21 wherein the autobiographical information is organized into a set of categories, said set of categories comprising life stats; religious or cultural health beliefs; diet and exercise profile; medications; lab tests; major social and life milestone events; medical visit findings; medical imaging; and diary.

29. (canceled)

30. (canceled)
